(19) United States
(12) Patent Application Publication
(10) Pub. No.: US 2022/0267169 A1
(43) Pub. Date: Aug. 25, 2022

(54) Intelligent Detection System of Effluent Total Nitrogen based on Fuzzy Transfer Learning Algorithm

(71) Applicant: BEIJING UNIVERSITY OF TECHNOLOGY, Beijing (CN)

(72) Inventors: Honggui Han, BEIJING (CN); Hongxu Liu, BEIJING (CN); Junfei Qiao, BEIJING (CN)

(21) Appl. No.: 17/676,692

(22) Filed: Feb. 21, 2022

(30) Foreign Application Priority Data
Feb. 22, 2021 (CN) 202110196095.9

(51) Int. Cl. C02F 1/00; G06N 3/04; G06N 3/08

(52) U.S. Cl. CPC C02F 1/008; G06N 3/0436; G06N 3/08; C02F 2209/14; C02F 2209/15; C02F 2209/10; C02F 2209/08; C02F 2209/006

(57) ABSTRACT

An intelligent detection system of effluent total nitrogen (TN) based on fuzzy transfer learning algorithm belongs to the field of intelligent detection technology. To detect the TN concentration, the artificial neural network can be used to model wastewater treatment process due to the nonlinear approximation ability and learning ability. However, wastewater treatment process has the characteristic of time-varying dynamics and external disturbance, artificial neural network prediction method cannot acquire sufficient data to ensure the accuracy of TN prediction, and data loss and data deficiency will make the prediction model invalid. The invention proposed an intelligent detection system of effluent total nitrogen based on fuzzy transfer learning algorithm; the proposed system contains several functional modules, including detection instrument, data acquisition, data storage and TN prediction. For the TN prediction module, the fuzzy transfer learning algorithm build the fuzzy neural network based intelligent prediction model, which the parameters are adjusted by the transfer learning method.

### CROSS-REFERENCE TO RELATED APPLICATION

This application claims the priority benefits to Chinese Patent Application No. 202110196095.9 filed on Feb. 22, 2021, the content of which is hereby incorporated by reference in its entirety.

### TECHNOLOGY AREA

Based on the operating characteristics of wastewater treatment process and using transfer learning based fuzzy neural network, the invention designs an intelligent detection method to realize the real-time measurement of effluent total nitrogen (TN) in wastewater treatment process. The concentration of effluent TN is the sum of all the nitrogen-containing pollutants in the effluent treated by the process facilities of the sewage treatment plant. It is an important indicator of water quality and the most important symbol of eutrophication of water body. The prediction of effluent TN has a great significance for realizing the monitoring and controlling in wastewater treatment process. It has great important influence for water quality standards and safety operation to apply intelligent recognition method in wastewater treatment system. Moreover, the above methods belonging to the control field and water treatment field simultaneously, is the important branch for the field of advanced manufacturing technology. Therefore, the intelligent detection of effluent TN is of great significance in the wastewater treatment system.

### TECHNOLOGY BACKGROUND

With the acceleration of urbanization process, the demand of freshwater resources is increased in china, which results in an increasing amount of wastewater. Therefore, wastewater treatment has become one of the important tasks in recent years. From the 1980s, large-scale construction of wastewater treatment plants began in China. Up to now, wastewater treatment capacity has been greatly improved, which has played a positive role in the prevention of water pollution and environmental protection.

The improvement of sewage treatment technology curbs the pollution of organic matter in wastewater, but the excessive discharge of nitrogen, phosphorus and other nutrients is still very serious. Among them, the TN concentration is the basic index to measure the effluent quality of wastewater treatment plants. The increase of nitrogen concentration is one of the main factors causing the deterioration of water quality and eutrophication. Currently, chemical experiments are used to predict TN concentration in wastewater treatment plants. Although the chemical method can guarantee the prediction accuracy, it has high requirements on the operating environment and long prediction time, which cannot meet the requirements of real-time prediction. In recent years, on-line instrument prediction can realize automatic prediction of nitrogen concentration, but the cost of instrument purchase and maintenance is high. Therefore, how to use information technology to achieve low cost and high precision forecast is important. Due to the nonlinear approximation ability and learning ability of artificial neural network, the wastewater treatment process can be effectively modeled, which provides a new method for the prediction of effluent water quality. However, wastewater treatment process has the characteristic of time-varying dynamics and external disturbance, artificial neural network prediction method cannot acquire the sufficient data to ensure the accuracy of TN prediction, and data loss and data deficiency will make the prediction model invalid. Therefore, the study of a new technology to solve the problem of high-precision measurement under data insufficiency has become an important topic in the field of wastewater control engineering research, and has important practical significance.

The invention proposed an intelligent detection system of effluent total nitrogen based on fuzzy transfer learning algorithm; the proposed system contains several functional modules, including detection instrument, data acquisition, data storage and TN prediction. For the TN prediction module, the fuzzy transfer learning algorithm build the fuzzy neural network based intelligent prediction model, which the parameters are adjusted by the transfer learning method. This algorithm can make use of the historical prediction knowledge of TN to make up for the deficiency of the current prediction data. Moreover, the intelligent prediction method also reduces the measurement cost, increase the prediction accuracy and improve the benefit of the wastewater treatment plant.

### SUMMARY

The invention proposed an intelligent detection system of effluent total nitrogen based on fuzzy transfer learning algorithm; the proposed system designs the fuzzy transfer learning algorithm for TN prediction and package it in module; Frist, the algorithm analyze the wastewater treatment process and select a group of auxiliary variable which is closely related to the TN, and use the fuzzy neural network to establish the reference model and prediction model of effluent TN. Then, the parameter knowledge is obtained through the reference model and the particle filter algorithm is designed to correct the parameter knowledge. In the end, the parameters of the prediction model are adjusted by using the parameter knowledge and data of the wastewater treatment process. The invention achieves accurate prediction of TN of effluent water, solves the problem of poor generalization ability of traditional fuzzy neural network in the case of insufficient data, and has good learning efficiency and prediction accuracy.

The invention includes the following technical solution and steps:

1. An intelligent detection system of effluent total nitrogen based on fuzzy transfer learning algorithm, its characteristic is that

The hardware includes several functional modules, including detection instrument, data acquisition, data storage and TN prediction; the specific implementation is as follows

The detection instrument contains ammonia nitrogen (NH₄-N) detector, nitrate nitrogen (NO₃-N) detector, suspended solids (SS) concentration detector, biochemical oxygen demand (BOD) detector and total phosphorus (TP) detector; the detection instruments are connected with programmable logic controller;

the programmable logic controller is connected with data processing module by the fieldbus; the variables of wastewater treatment process are analyzed by Principal Component Analysis, and the input variables of TN prediction model are selected as: NH₄-N, NO₃-N, SS, BOD, TP, The output value of TN prediction model is the TN values;

the data processing module is connected with data storage module by the fieldbus;

the data storage module is connected with TN prediction module using communication interface

The TN prediction module comprise the following steps:

(1) Establish Prediction Model of Effluent TN Based on Fuzzy-Neural-Network

The structure of fuzzy-neural-network contains four layers: input layer, membership function layer, rule layer and output layer, the network is 5-10-10-1, including 5 neurons in input layer, 10 neurons in membership function layer, 10 neurons in rule layer and 1 neurons in output layer; connecting weights between input layer and membership function layer are assigned as 1, connecting weights between membership layer and rule function layer are assigned 1, connecting weights between rule layer and output layer are randomly initialized in [−1, 1]; the number of the training sample of prediction model is T, the input of fuzzy-neural-network prediction model is o(t)=[o¹(t), o²(t), o³(t), o⁴(t), o⁵(t)] at time t, o¹(t) represents NH₄-N concentration at time t; o²(t) represents NO₃-N concentration at time t, o³(t) represents SS concentration at time t, o⁴(t) represents BOD value at time t, and o⁵(t) represents TP concentration at time t, the output of fuzzy neural network is y(t) and the actual output is y_{d}(t); fuzzy-neural-network prediction model includes:

1) Input layer: there are 5 neurons in this layer, the output is:

x^{p}(t)=o^{p}(t), (1)

where x^{p}(t) is the pth output value of input neuron at time t, t=1, . . . , T, p=1, . . . , 5,

2) Membership function layer: there are 10 neurons in membership function layer, the output of membership function neuron is:

$${φ}^{k} ( t ) = {\prod }_{p = 1}^{5} exp ( - \frac{{( {x}^{p} ( t ) - {c}^{p k} ( t ) )}^{2}}{2 {( {σ}^{p k} ( t ) )}^{2}} ) , \qquad ( 2 )$$

where φ^{k}(t) is the kth output value of membership function neuron at time t, k=1, . . . , 10, c^{pk}(t) is the pth center of the kth membership function neuron at time t, which is randomly initialized in [−1, 1]; σ^{pk}(t) is the pth width of the kth membership function neuron at time t, which is randomly initialized in [−1, 1];

3) Rule layer: there are 10 neurons in this layer, and the output of rule neuron is:

$${v}^{k} ( t ) = \frac{{φ}^{k} ( t )}{{\sum }_{k = 1}^{10} {φ}^{k} ( t )} , \qquad ( 3 )$$

where v^{k}(t) is the kth output value of rule neuron at time t;

4) Output layer: the output of output neuron is:

$$y ( t ) = {\sum }_{k = 1}^{10} {w}^{k} ( t ) {v}^{k} ( t ) , \qquad ( 4 )$$

where y(t) is the output of fuzzy-neural-network prediction model at time t, w^{k}(t) is the connecting weight between kth rule neuron and output neuron;

(2) Establish the reference model of effluent TN to acquire knowledge

The structure of reference model is same as the prediction model, the number of the training sample of reference model is N;

1) Construct the reference model:

$${y}_{Z} ( n ) = {\sum }_{k = 1}^{10} {w}_{Z}^{k} ( n ) \frac{{\prod }_{p = 1}^{5} exp ( - \frac{{( {x}_{Z}^{p} ( n ) - {c}_{Z}^{p k} ( n ) )}^{2}}{2 {( {σ}_{Z}^{p k} ( n ) )}^{2}} )}{{\sum }_{k = 1}^{10} {\prod }_{p = 1}^{5} exp ( - \frac{{( {x}_{Z}^{p} ( n ) - {c}_{Z}^{p k} ( n ) )}^{2}}{2 {( {σ}_{Z}^{p k} ( n ) )}^{2}} )} , \qquad ( 5 )$$

where y_{Z}(n) is the output of fuzzy neural network reference model at time n, n=1, . . . , N,w_{Z}^{k}(n) is the connecting weight between kth rule neuron and output neuron at time n, which is randomly initialized in [0, 1], c_{Z}^{pk} (n) is the pth center of the kth membership function neuron at time n, which is randomly initialized in [−1, 1], d_{Z}^{pk} (n) is the pth width of the kth membership function neuron at time n, which is randomly initialized in [−1, 1];

2) Leverage gradient descent algorithm to train the reference model; the center, width and weight of the reference model are updated as:

$$E ( n ) = \frac{1}{2} {( {y}_{Z} ( n ) - {y}_{Z d} ( n ) )}^{2} , \qquad ( 6 )$$

$${c}_{Z}^{p k} ( n + 1 ) = {c}_{Z}^{p k} ( n ) - λ \frac{\partial E ( n )}{\partial {c}_{Z}^{p k} ( n )} , \qquad ( 7 )$$

$${σ}_{Z}^{p k} ( n + 1 ) = {σ}_{Z}^{p k} ( n ) - λ \frac{\partial E ( n )}{\partial {σ}_{Z}^{p k} ( n )} , \qquad ( 8 )$$

$${w}_{Z}^{k} ( n + 1 ) = {w}_{Z}^{k} ( n ) - λ \frac{\partial E ( n )}{\partial {w}_{Z}^{k} ( n )} , \qquad ( 9 )$$

where E(n) is the objection function of reference model at time n, y_{Zd}(n) is the desired output of reference model at time n, 2 is the learning rate of gradient descent algorithm, which is randomly initialized in [0;01, 0;1];

3) Compute E(n+1) using equation (6), if n<N or E(n+1)>0;01, n=n+1, go to step \hat{2}; else stop the training process, given c_{Z}^{pk}(n), σ_{Z}^{pk}(n), w_{Z}^{k}(n) to the reference model;

4) Acquire parameter knowledge from the reference model; the parameter knowledge can be given as

k_{Z}^{k}(n)=[c_{Z}^{1k}(n), . . . , c_{Z}^{Pk}(n), σ_{Z}^{1k}(n), . . . , σ_{Z}^{Pk}(n), w_{Z}^{k}(n)]^{T}, (10)

where k_{Z}^{k}(n) is the kth parameter knowledge extracted from reference model at time n, K_{Z}(n)=[k¹_{Z}(n)^{T}, . . . , k¹⁰_{Z}(n)^{T}]^{T} is the parameter knowledge extracted from reference model at time n;

(3) Leverage parameter knowledge and data to train prediction model;

1) Adjust parameter knowledge using particle filter algorithm; particle filter algorithm consists of three steps: knowledge sampling, knowledge evaluation and knowledge fusion; the knowledge sampling process is

Kₗ(t)=K_{Z}(n)+δₗ(t), (11)

where Kₗ(t) is the lth sampling knowledge, l=1, . . . , L, L=30 is the number of sampling, δₗ(t) is the random sampling vector, which is randomly initialized in [0, 1]; knowledge evaluation includes two indexes of knowledge matching degree and knowledge diversity, which are expressed as

ωₗ(t)=1−e^{-(D}^{ₗ}^{(t)+M}^{ₗ}^{(t))}^{²}, (12)

Mₗ(t)=e^{-(y(K}^{ₗ}^{(t),o(t))-y}^{_{d}}^{(t))}^{²}, (13)

Dₗ(t)=e^{-cos(K}^{ₗ}^{(t),K}^{_{Z}}⁽ⁿ⁾⁾, (14)

where ωₗ(t) is the importance weight of the lth sampling knowledge at time t, Mₗ(t) is the knowledge matching degree between lth sampling knowledge and the training data at time t, y(Kₗ(t), o(t)) is the output of prediction model with Kₗ(t) as parameter at time t, y_{d}(t) is the desired output of prediction model at time t, Dₗ(t) is the knowledge diversity of lth sampling knowledge at time t; based on the sampling knowledge and importance weight, the knowledge fusion process can be expressed as

$${K}_{R} ( t ) = {\sum }_{l = 1}^{L} {K}_{l} ( t ) \cdot {ω}_{l} ( t ) , \qquad ( 15 )$$

where K_{R}(t)=[k_{R}¹(t)^{T}, . . . , k_{R}¹⁰(t)^{T}]^{T} is the reconstruction knowledge at time t, k_{R}^{k} (t) is the kth reconstruction knowledge

k_{R}^{k}(t)=[c_{R}^{1k}(t), . . . , c_{R}^{pk}(t), . . . , c_{R}^{Pk}(t), σ_{R}^{1k}(t), . . . , σ_{R}^{pk}(t), . . . , σ_{R}^{Pk}(t), w_{R}^{k}(t)]^{T}. (16)

2) Leverage reconstruction knowledge and data to adjust the parameters of prediction model; the knowledge and data driven objective function of the prediction model is

$${E}^{K D} ( t ) = α ( t ) {e ( t )}^{2} + β ( t ) {\sum }_{k = 1}^{10} {( {w}^{k} ( t ) - {w}_{R}^{k} ( t ) )}^{2} {\sum }_{p = 1}^{5} {( {c}^{p k} ( t ) - {c}_{R}^{p k} ( t ) )}^{2} + {( {σ}^{p k} ( t ) - {σ}_{R}^{p k} ( t ) )}^{2} , \qquad ( 17 )$$

where E^{KD}(t) is the objection function of prediction model at time t, e(t)=y(t)−y_{d}(t) is the output error of prediction model at time t; α(t)∈(0;5, 1] and β(t)c (0, 0;1] are balancing parameter, the updating process of c^{pk}(t), σ^{pk}(t), w^{k}(t), α(t) and β(t) are

$${c}^{p k} ( t + 1 ) = {c}^{p k} ( t ) - λ \frac{\partial {E}^{K D} ( t )}{\partial {c}^{p k} ( t )} , \qquad ( 18 )$$

$${σ}^{p k} ( t + 1 ) = {σ}^{p k} ( t ) - λ \frac{\partial {E}^{K D} ( t )}{\partial {σ}^{p k} ( t )} , \qquad ( 19 )$$

$${w}^{k} ( t + 1 ) = {w}^{k} ( t ) - λ \frac{\partial {E}^{K D} ( t )}{\partial {w}^{k} ( t )} , \qquad ( 20 )$$

$$α ( t + 1 ) = α ( t ) - λ \frac{\partial {E}^{K D} ( t )}{\partial α ( t )} , \qquad ( 21 )$$

$$β ( t + 1 ) = β ( t ) - λ \frac{\partial {E}^{K D} ( t )}{\partial β ( t )} , \qquad ( 22 )$$

3) Compute E^{KD}(t+1) using equation (17), if t<Q or E^{KD}(t+1)>0.01, t=t+1, go to step 2); else stop the training process, given c^{pk}(t), σ^{pk}(t), w^{k}(t) to the prediction model;

(4) Effluent total nitrogen TN concentration prediction;

The number of the testing samples is M; the testing samples are used as the input of prediction model, the output of prediction model is the soft-computing values of TN concentration.

The novelties of this invention contain:

(1) Aiming at the problem that it is difficult to train and obtain an accurate total nitrogen prediction model in the case of insufficient data, a fuzzy transfer-based intelligent prediction method is proposed. This method establishes a prediction model based on fuzzy neural network and uses the knowledge and data to adjust the parameters of the prediction model to make up for the shortcomings of the current lack of data;

(2) Aiming at the problem that the history knowledge does not completely conform to the current prediction task, a knowledge reconstruction mechanism based on particle filter algorithm is proposed, which uses the current prediction data to correct the historical knowledge and increases the validity of the knowledge;

(3) The invention designs a new objective function, which sets balancing parameter for knowledge-driven terms and data-driven terms to avoids the problems of knowledge over-fitting and data over-fitting, and adopts gradient descent algorithm to optimize network parameters online and improve the accuracy of prediction model.

### DESCRIPTION OF DRAWINGS

FIG. 1 is the structure of intelligent detection system

FIG. 2 is the algorithm of fuzzy transfer algorithm

FIG. 3 is the structure of fuzzy neural network

FIG. 4 is the prediction result diagram of the TN concentration

FIG. 5 is the prediction error diagram of the TN concentration

### DETAILED DESCRIPTION OF THE INVENTION

The experimental data come from the 2017 water quality data analysis report of a wastewater treatment plant. Where the actual testing data about NH₄-N, NO₃-N, SS, BOD, TP are selected for the experimental sample data. There are 400 groups data are available after eliminate the abnormal, where 5000 history samples are selected as the reference dataset and 4000 current samples are selected as the prediction dataset. For the prediction dataset, 2000 samples are used for training prediction model and 2000 samples are used for testing prediction model.

1. An intelligent detection system of effluent total nitrogen (TN) based on fuzzy transfer learning algorithm, its characteristic is that

The hardware includes several functional modules, including detection instrument, data acquisition, data processing, data storage and TN prediction, which can be seen in FIG. 1;

the detection instrument contains ammonia nitrogen (NH₄-N) detector, nitrate nitrogen (NO₃-N) detector, suspended solids (SS) concentration detector, biochemical oxygen demand (BOD) detector and total phosphorus (TP) detector; the detection instruments are connected with programmable logic controller to realize data acquiring;

the programmable logic controller is connected with data processing module to realize feature selection of TN; wherein the data processing module use Principal Component Analysis to select a group of auxiliary variable which is closely related to the TN; the input variables of TN prediction model are selected as: NH₄-N, NO₃-N, SS, BOD, TP, The output value of TN prediction model is the TN values, the units are mg/l;

the data processing module is connected with data storage module;

the data storage module is connected with TN prediction module using communication interface;

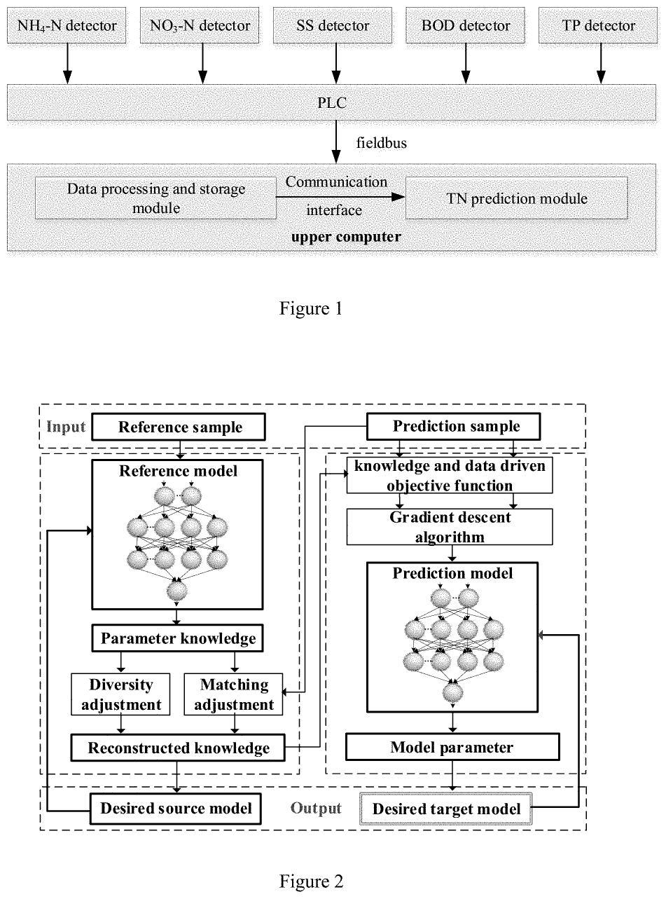

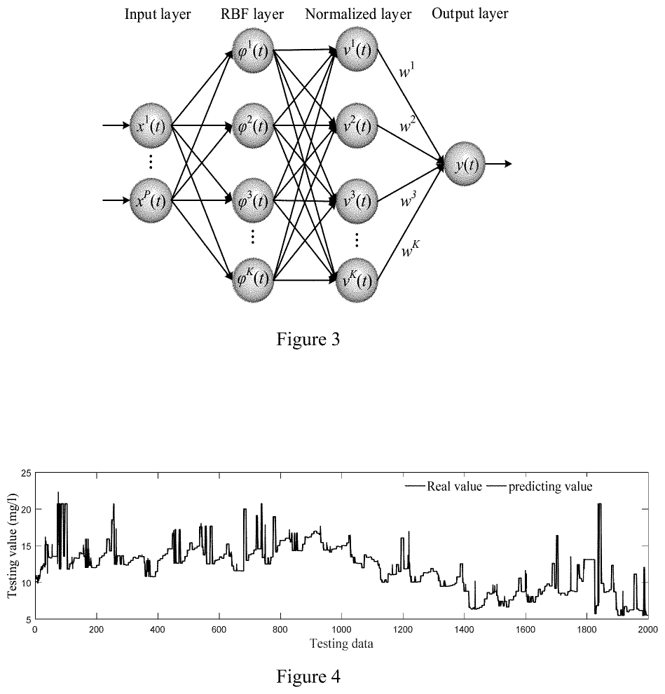

the TN prediction module comprise the following steps: first, the algorithm use the fuzzy neural network to establish the reference model and prediction model of effluent TN. Then, the parameter knowledge is obtained through the reference model and the particle filter algorithm is designed to correct the parameter knowledge. In the end, the parameters of the prediction model are adjusted by using the parameter knowledge and data of the wastewater treatment process to realize online TN prediction, which can be seen in FIG. 2;

(1) Establish Prediction Model of Effluent TN Based on Fuzzy-Neural-Network

The structure of fuzzy-neural-network contains four layers: input layer, membership function layer, rule layer and output layer, which can be seen in FIG. 3; the network is 5-10-10-1, including 5 neurons in input layer, 10 neurons in membership function layer, 10 neurons in rule layer and 1 neurons in output layer; connecting weights between input layer and membership function layer are assigned as 1, connecting weights between membership layer and rule function layer are assigned 1, connecting weights between rule layer and output layer are randomly initialized in [−1, 1]; the number of the training sample of prediction model is T, the input of fuzzy-neural-network prediction model is o(t)=[o¹(t), o²(t), o³(t), o⁴(t), o⁵(t)] at time t, o¹(t) represents NH₄-N concentration at time t; o²(t) represents NO₃-N concentration at time t, o³(t) represents SS concentration at time t, o⁴(t) represents BOD value at time t, and o⁵(t) represents TP concentration at time t, the output of fuzzy neural network is y(t) and the actual output is y_{d}(t); fuzzy-neural-network prediction model includes:

1) Input layer: there are 5 neurons in this layer, the output is:

x^{p}(t)=o^{p}(t), (1)

where x^{p}(t) is the pth output value of input neuron at time t, t=1, . . . , T, p=1, . . . , 5,

2) Membership function layer: there are 10 neurons in membership function layer, the output of membership function neuron is:

$${φ}^{k} ( t ) = {\prod }_{p = 1}^{5} exp ( - \frac{{( {x}^{p} ( t ) - {c}^{p k} ( t ) )}^{2}}{2 {( {σ}^{p k} ( t ) )}^{2}} ) , \qquad ( 2 )$$

where φ^{k}(t) is the kth output value of membership function neuron at time t, k=1, . . . , 10, c^{pk}(t) is the pth center of the kth membership function neuron at time t, which is randomly initialized in [−1, 1]; σ^{pk}(t) is the pth width of the kth membership function neuron at time t, which is randomly initialized in [−1, 1];

3) Rule layer: there are 10 neurons in this layer, and the output of rule neuron is:

$${v}^{k} ( t ) = \frac{{φ}^{k} ( t )}{{\sum }_{k = 1}^{10} {φ}^{k} ( t )} , \qquad ( 3 )$$

where v^{k}(t) is the kth output value of rule neuron at time t;

4) Output layer: the output of output neuron is:

$$y ( t ) = {\sum }_{k = 1}^{10} {w}^{k} ( t ) {v}^{k} ( t ) , \qquad ( 4 )$$

where y(t) is the output of fuzzy-neural-network prediction model at time t, w^{k}(t) is the connecting weight between kth rule neuron and output neuron;

(2) Establish the reference model of effluent TN to acquire knowledge

The structure of reference model is same as the prediction model, the number of the training sample of reference model is N;

1) Construct the reference model:

$${y}_{Z} ( n ) = {\sum }_{k = 1}^{10} {w}_{Z}^{k} ( n ) \frac{{\prod }_{p = 1}^{5} exp ( - \frac{{( {x}_{Z}^{p} ( n ) - {c}_{Z}^{p k} ( n ) )}^{2}}{2 {( {σ}_{Z}^{p k} ( n ) )}^{2}} )}{{\sum }_{k = 1}^{10} {\prod }_{p = 1}^{5} exp ( - \frac{{( {x}_{Z}^{p} ( n ) - {c}_{Z}^{p k} ( n ) )}^{2}}{2 {( {σ}_{Z}^{p k} ( n ) )}^{2}} )} , \qquad ( 5 )$$

where y_{Z}(n) is the output of fuzzy neural network reference model at time n, n=1, . . . , N, w_{Z}^{k}(n) is the connecting weight between kth rule neuron and output neuron at time n, which is randomly initialized in [0, 1], c_{Z}^{pk}(n) is the pth center of the kth membership function neuron at time n, which is randomly initialized in [−1, 1], σ_{Z}^{pk}(n) is the pth width of the kth membership function neuron at time n, which is randomly initialized in [−1, 1];

2) Leverage gradient descent algorithm to train the reference model; the center, width and weight of the reference model are updated as:

$$E ( n ) = \frac{1}{2} {( {y}_{Z} ( n ) - {y}_{Z d} ( n ) )}^{2} , \qquad ( 6 )$$

$${c}_{Z}^{p k} ( n + 1 ) = {c}_{Z}^{p k} ( n ) - λ \frac{\partial E ( n )}{\partial {c}_{Z}^{p k} ( n )} , \qquad ( 7 )$$

$${σ}_{Z}^{p k} ( n + 1 ) = {σ}_{Z}^{p k} ( n ) - λ \frac{\partial E ( n )}{\partial {σ}_{Z}^{p k} ( n )} , \qquad ( 8 )$$

$${w}_{Z}^{k} ( n + 1 ) = {w}_{Z}^{k} ( n ) - λ \frac{\partial E ( n )}{\partial {w}_{Z}^{k} ( n )} , \qquad ( 9 )$$

where E(n) is the objection function of reference model at time n, y_{Zd}(n) is the desired output of reference model at time n, λ is the learning rate of gradient descent algorithm, which is randomly initialized in [0;01, 0;1];

3) Compute E(n+1) using equation (6), if n<N or E(n+1)>0;01, n=n+1, go to step \hat{2}; else stop the training process, given c_{Z}^{pk}(n), σ_{Z}^{pk}(n), w_{Z}^{k}(n) to the reference model;

4) Acquire parameter knowledge from the reference model; the parameter knowledge can be given as

k_{Z}^{k}(n)=[c_{Z}^{1k}(n), . . . , c_{Z}^{Pk}(n), σ_{Z}^{1k}(n), . . . , σ_{Z}^{Pk}(n), w_{Z}^{k}(n)]^{T}, (10)

where k_{Z}^{k}(n) is the kth parameter knowledge extracted from reference model at time n, K_{Z}(n)=[k_{Z}¹(n)^{T}, . . . , k_{Z}¹⁰(n)^{T}]^{T} is the parameter knowledge extracted from reference model at time n;

(3) Leverage parameter knowledge and data to train prediction model;

1) Adjust parameter knowledge using particle filter algorithm; particle filter algorithm consists of three steps: knowledge sampling, knowledge evaluation and knowledge fusion; the knowledge sampling process is

Kₗ(t)=K_{Z}(n)+δₗ(t), (11)

where Kₗ(t) is the lth sampling knowledge, l=1, . . . , L, L=30 is the number of sampling, δₗ(t) is the random sampling vector, which is randomly initialized in [0, 1]; knowledge evaluation includes two indexes of knowledge matching degree and knowledge diversity, which are expressed as

ωₗ(t)=1−e^{-(D}^{ₗ}^{(t)+M}^{ₗ}^{(t))}^{²}, (12)

Mₗ(t)=e^{-(y(K}^{ₗ}^{(t),o(t))-y}^{_{d}}^{(t))}^{²}, (13)

Dₗ(t)=e^{-cos(K}^{ₗ}^{(t),K}^{_{Z}}⁽ⁿ⁾⁾, (14)

where ωₗ(t) is the importance weight of the lth sampling knowledge at time t, Mₗ(t) is the knowledge matching degree between lth sampling knowledge and the training data at time t, y(Kₗ(t), o(t)) is the output of prediction model with Kₗ(t) as parameter at time t, y_{d}(t) is the desired output of prediction model at time t, Dₗ(t) is the knowledge diversity of lth sampling knowledge at time t; based on the sampling knowledge and importance weight, the knowledge fusion process can be expressed as

$${K}_{R} ( t ) = {{\sum }_{l = 1}}^{L} {K}_{l} ( t ) \cdot {ω}_{l} ( t ) , \qquad ( 15 )$$

where K_{R}(t)=[k_{R}¹(t)^{T}, . . . , k_{R}¹⁰(t)^{T}]^{T} is the reconstruction knowledge at time t, k_{R}^{k}(t) is the kth reconstruction knowledge

k_{R}^{k}(t)=[c_{R}^{1k}(t), . . . , c_{R}^{pk}(t), . . . , c_{R}^{Pk}(t), σ_{R}^{1k}(t), . . . , σ_{R}^{pk}(t), . . . , σ_{R}^{Pk}(t), w_{R}^{k}(t)]^{T}. (16)

2) Leverage reconstruction knowledge and data to adjust the parameters of prediction model; The objective function of the prediction model is

$${E}^{K D} ( t ) = α ( t ) {e ( t )}^{2} + β ( t ) {\sum }_{k = 1}^{10} {( {w}^{k} ( t ) - {w}_{R}^{k} ( t ) )}^{2} {\sum }_{p = 1}^{5} {( {c}^{pk} ( t ) - {c}_{R}^{pk} ( t ) )}^{2} + {( {σ}^{pk} ( t ) - {σ}_{R}^{pk} ( t ) )}^{2} , \qquad ( 17 )$$

where E^{KD}(t) is the objection function of prediction model at time t, e(t)=y(t)−y_{d}(t) is the output error of prediction model at time t; α(t)∈(0;5, 1] and β(t)∈(0, 0;11 are balancing parameter, the updating process of c^{pk}(t), σ^{pk}(t), w^{k}(t), α(t) and β(t) are

$${c}^{p k} ( t + 1 ) = {c}^{p k} ( t ) - λ \frac{\partial {E}^{K D} ( t )}{\partial {c}^{p k} ( t )} , \qquad ( 18 )$$

$${σ}^{p k} ( t + 1 ) = {σ}^{p k} ( t ) - λ \frac{\partial {E}^{K D} ( t )}{\partial {σ}^{p k} ( t )} , \qquad ( 19 )$$

$${w}^{k} ( t + 1 ) = {w}^{k} ( t ) - λ \frac{\partial {E}^{K D} ( t )}{\partial {w}^{k} ( t )} , \qquad ( 20 )$$

$$α ( t + 1 ) = α ( t ) - λ \frac{\partial {E}^{K D} ( t )}{\partial α ( t )} , \qquad ( 21 )$$

$$β ( t + 1 ) = β ( t ) - λ \frac{\partial {E}^{K D} ( t )}{\partial β ( t )} , \qquad ( 22 )$$

3) Compute E^{KD}(t+1) using equation (17), if t<Q or E^{KD}(t+1)>0.01, t=t+1, go to step 2); else stop the training process, given c^{pk}(t), σ^{pk}(t), w^{k}(t) to the prediction model;

(4) Effluent total nitrogen TN concentration prediction;

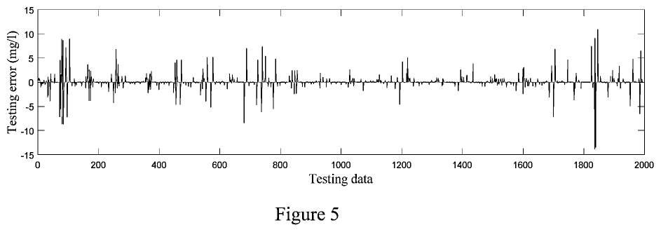

The number of the testing samples is M; the testing samples are used as the input of prediction model, the output of prediction model is the soft-computing values of TN concentration; the testing results are shown in FIG. 4 and FIG. 5, FIG. 4 is the prediction result diagram of the TN concentration, FIG. 5 is the prediction error diagram of the TN concentration.

1. An intelligent detection system of effluent total nitrogen (TN) based on fuzzy transfer learning algorithm, its characteristic is that
The detection instrument contains ammonia nitrogen (NH₄-N) detector, nitrate nitrogen (NO₃-N) detector, suspended solids (SS) concentration detector, biochemical oxygen demand (BOD) detector and total phosphorus (TP) detector; the detection instruments are connected with programmable logic controller;
the programmable logic controller is connected with data processing module by the fieldbus; the variables of wastewater treatment process are analyzed by Principal Component Analysis, and the input variables of TN prediction model are selected as: NH₄-N, NO₃-N, SS, BOD, TP, The output value of TN prediction model is the TN values;
the data processing module is connected with data storage module by the fieldbus;
the data storage module is connected with TN prediction module using communication interface;
The TN prediction module comprise the following steps:
(1) Establish prediction model of effluent TN based on fuzzy-neural-network
The structure of fuzzy-neural-network contains four layers: input layer, membership function layer, rule layer and output layer, the network is 5-10-10-1, including 5 neurons in input layer, 10 neurons in membership function layer, 10 neurons in rule layer and 1 neurons in output layer; connecting weights between input layer and membership function layer are assigned as 1, connecting weights between membership layer and rule function layer are assigned 1, connecting weights between rule layer and output layer are randomly initialized in [−1, 1]; the number of the training sample of prediction model is T, the input of fuzzy-neural-network prediction model is o(t)=[o¹(t), o²(t), o³(t), o⁴(t), o⁵ (t)] at time t, o^{l}(t) represents NH₄-N concentration at time t; o²(t) represents NO₃-N concentration at time t, o³(t) represents SS concentration at time t, o⁴(t) represents BOD value at time t, and o⁵(t) represents TP concentration at time t, the output of fuzzy neural network is y(t) and the actual output is y_{d}(t); fuzzy-neural-network prediction model includes:
1) Input layer: there are 5 neurons in this layer, the output is:
x^{p}(t)=o^{p}(t), (1)
where x^{p}(t) is the pth output value of input neuron at time t, t=1, T, p=1, . . . , 5,
2) Membership function layer: there are 10 neurons in membership function layer, the output of membership function neuron is:
$${φ}^{k} ( t ) = {\prod }_{p = 1}^{5} exp ( - \frac{{( {x}^{p} ( t ) - {c}^{p k} ( t ) )}^{2}}{2 {( {σ}^{p k} ( t ) )}^{2}} ) , \qquad ( 2 )$$
where ϕ^{k}(t) is the kth output value of membership function neuron at time t, k=1, . . . , 10, c^{pk}(t) is the pth center of the kth membership function neuron at time t, which is randomly initialized in [−1, 1]; σ^{pk}(t) is the pth width of the kth membership function neuron at time t, which is randomly initialized in [−1, 1];
3) Rule layer: there are 10 neurons in this layer, and the output of rule neuron is:
$${v}^{k} ( t ) = \frac{{φ}^{k} ( t )}{{\sum }_{k = 1}^{10} {φ}^{k} ( t )} , \qquad ( 3 )$$
where v^{k}(t) is the kth output value of rule neuron at time t;
4) Output layer: the output of output neuron is:
$$y ( t ) = {\sum }_{k = 1}^{10} {w}^{k} ( t ) {v}^{k} ( t ) , \qquad ( 4 )$$
where y(t) is the output of fuzzy-neural-network prediction model at time t, w^{k}(t) is the connecting weight between kth rule neuron and output neuron;
(2) Establish the reference model of effluent TN to acquire knowledge
The structure of reference model is same as the prediction model, the number of the training sample of reference model is N;
1) Construct the reference model:
$${y}_{Z} ( n ) = {\sum }_{k = 1}^{10} {w}_{Z}^{k} ( n ) \frac{{\prod }_{p = 1}^{5} exp ( - \frac{{( {x}_{Z}^{p} ( n ) - {c}_{Z}^{p k} ( n ) )}^{2}}{2 {( {σ}_{Z}^{p k} ( n ) )}^{2}} )}{{\sum }_{k = 1}^{10} {\prod }_{p = 1}^{5} exp ( - \frac{{( {x}_{Z}^{p} ( n ) - {c}_{Z}^{p k} ( n ) )}^{2}}{2 {( {σ}_{Z}^{p k} ( n ) )}^{2}} )} , \qquad ( 5 )$$
where y_{Z}(n) is the output of fuzzy neural network reference model at time n, n=1, . . . , N,w_{Z}^{k}(n) is the connecting weight between kth rule neuron and output neuron at time n, which is randomly initialized in [0, 1], c_{Z}^{pk}(n) is the pth center of the kth membership function neuron at time n, which is randomly initialized in [−1, 1], σ_{Z}^{pk}(n) is the pth width of the kth membership function neuron at time n, which is randomly initialized in [−1, 1];
2) Leverage gradient descent algorithm to train the reference model; the center, width and weight of the reference model are updated as:
$$E ( n ) = \frac{1}{2} {( {y}_{Z} ( n ) - {y}_{Z d} ( n ) )}^{2} , \qquad ( 6 )$$
$${c}_{Z}^{p k} ( n + 1 ) = {c}_{Z}^{p k} ( n ) - λ \frac{\partial E ( n )}{\partial {c}_{Z}^{p k} ( n )} , \qquad ( 7 )$$
$${σ}_{Z}^{p k} ( n + 1 ) = {σ}_{Z}^{p k} ( n ) - λ \frac{\partial E ( n )}{\partial {σ}_{Z}^{p k} ( n )} , \qquad ( 8 )$$
$${w}_{Z}^{k} ( n + 1 ) = {w}_{Z}^{k} ( n ) - λ \frac{\partial E ( n )}{\partial {w}_{Z}^{k} ( n )} , \qquad ( 9 )$$
where E(n) is the objection function of reference model at time n, y_{Zd}(n) is the desired output of reference model at time n, 2 is the learning rate of gradient descent algorithm, which is randomly initialized in [0;01, 0;1];
3) Compute E(n+1) using equation (6), if n<N or E(n+1)>0;01, n=n+1, go to step \hat{2}; else stop the training process, given c_{Z}^{pk}(n), σ_{Z}^{pk}(n), w_{Z}^{k}(n) to the reference model;
4) Acquire parameter knowledge from the reference model; the parameter knowledge can be given as
k_{Z}^{k}(n)=[c_{Z}^{1k}(n), . . . , c_{Z}^{Pk}(n), σ_{Z}^{1k}(n), . . . , σ_{Z}^{Pk}(n), w_{Z}^{k}(n)]^{T}, (10)
where k_{Z}^{k}(n) is the kth parameter knowledge extracted from reference model at time n, K_{Z}(n)=[k_{Z}¹(n)^{T}, . . . , k_{Z}¹⁰(n)^{T}]^{T} is the parameter knowledge extracted from reference model at time n;
(3) Leverage parameter knowledge and data to train prediction model;
1) Adjust parameter knowledge using particle filter algorithm; particle filter algorithm consists of three steps: knowledge sampling, knowledge evaluation and knowledge fusion; the knowledge sampling process is
Kₗ(t)=K_{Z}(n)+δₗ(t), (11)
where Kₗ(t) is the lth sampling knowledge, l=1, . . . , L, L=30 is the number of sampling, δₗ(t) is the random sampling vector, which is randomly initialized in [0, 1]; knowledge evaluation includes two indexes of knowledge matching degree and knowledge diversity, which are expressed as
ωₗ(t)=1−e^{-(D}^{ₗ}^{(t)+M}^{ₗ}^{(t))}^{²}, (12)
Mₗ(t)=e^{-(y(K}^{ₗ}^{(t),o(t))-y}^{_{d}}^{(t))}^{²}, (13)
Dₗ(t)=e^{-cos(K}^{ₗ}^{(t),K}^{_{Z}}⁽ⁿ⁾⁾, (14)
where ω(t) is the importance weight of the lth sampling knowledge at time t, Mₗ(t) is the knowledge matching degree between lth sampling knowledge and the training data at time t, y(Kₗ(t), o(t)) is the output of prediction model with Kₗ(t) as parameter at time t, y_{d}(t) is the desired output of prediction model at time t, Dₗ(t) is the knowledge diversity of lth sampling knowledge at time t; based on the sampling knowledge and importance weight, the knowledge fusion process can be expressed as
$${K}_{R} ( t ) = {\sum }_{l = 1}^{L} {K}_{l} ( t ) \cdot {ω}_{l} ( t ) , \qquad ( 15 )$$
where K_{R}(t)=[k_{R}¹(t)^{T}, . . . , k_{R}¹⁰(t)^{T}]^{T} is the reconstruction knowledge at time t, k^{k}_{R} (t) is the kth reconstruction knowledge
k_{R}^{k}(t)=[c_{R}^{1k}(t), . . . , c_{R}^{pk}(t), . . . , c_{R}^{Pk}(t), σ_{R}^{1k}(t), . . . , σ_{R}^{pk}(t), . . . , σ_{R}^{Pk}(t), w_{R}^{k}(t)]^{T}. (16)
2) Leverage reconstruction knowledge and data to adjust the parameters of prediction model; The objective function of the prediction model is
$${E}^{K D} ( t ) = α ( t ) {e ( t )}^{2} + β ( t ) {\sum }_{k = 1}^{10} {( {w}^{k} ( t ) - {w}_{R}^{k} ( t ) )}^{2} {\sum }_{p = 1}^{5} {( {c}^{pk} ( t ) - {c}_{R}^{pk} ( t ) )}^{2} + {( {σ}^{pk} ( t ) - {σ}_{R}^{pk} ( t ) )}^{2} , \qquad ( 17 )$$
where E^{KD}(t) is the objection function of prediction model at time t, e(t)=y(t)−y_{d}(t) is the output error of prediction model at time t; α(t)∈(0;5, 1] and β(t)∈(0, 0;1] are balancing parameter, the updating process of c^{pk}(t), σ^{pk}(t), w^{k}(t), α(t) and β(t) are
$${c}^{p k} ( t + 1 ) = {c}^{p k} ( t ) - λ \frac{\partial {E}^{K D} ( t )}{\partial {c}^{p k} ( t )} , \qquad ( 18 )$$
$${σ}^{p k} ( t + 1 ) = {σ}^{p k} ( t ) - λ \frac{\partial {E}^{K D} ( t )}{\partial {σ}^{p k} ( t )} , \qquad ( 19 )$$
$${w}^{k} ( t + 1 ) = {w}^{k} ( t ) - λ \frac{\partial {E}^{K D} ( t )}{\partial {w}^{k} ( t )} , \qquad ( 20 )$$
$$α ( t + 1 ) = α ( t ) - λ \frac{\partial {E}^{K D} ( t )}{\partial α ( t )} , \qquad ( 21 )$$
$$β ( t + 1 ) = β ( t ) - λ \frac{\partial {E}^{K D} ( t )}{\partial β ( t )} , \qquad ( 22 )$$
3) Compute E^{KD}(t+1) using equation (17), if t<Q or E^{KD}(t+1)>0.01, t=t+1, go to step 2); else stop the training process, given c^{pk}(t), σ^{pk}(t), w^{k}(t) to the prediction model;
(4) Effluent total nitrogen TN concentration prediction;
The number of the testing samples is M; the testing samples are used as the input of prediction model, the output of prediction model is the soft-computing values of TN concentration.
